(19) United States
(12) Patent Application Publication
(10) Pub. No.: US 2009/0280574 A1
(43) Pub. Date: Nov. 12, 2009

(54) Apparatus for Indicating The Presence of a Controlled Substance and Method of Use

(76) Inventor: Wendi Bryant, Lakeworth, FL (US)

Correspondence Address: FRASCOGNA COURTNEY, PLLC, P.O.BOX 23126, JACKSON, MS, 39225-3126, US

(21) Appl. No.: 12/115,635

(22) Filed: May 6, 2008

(51) Int. Cl. G01N 33/00; B01J 19/00

(52) U.S. Cl. 436/183; 422/55

(57) ABSTRACT

An apparatus for indicating the presence of a controlled substance is disclosed for use in medical facilities licensed to administer controlled medications to patients. The apparatus contains an indicator element selected to experience a color changing chemical reaction when mixed with a specific type of medication. A medical professional can inject a portion of any unused medication into the apparatus prior to final disposal to prove that the disposed of medication is not some other compound.

### FIELD OF THE INVENTION

This invention relates generally to chemical analysis and specifically to drug testing systems for hospitals, pharmacies, and other facilities involved in handling and/or disposing of narcotics.

### BACKGROUND OF THE INVENTION

When a doctor prescribes medication for a patient in a medical facility, a medical professional is typically charged with administering the medication. Often, the medication arrives in a container that contains more medication than the prescribed dose. The policy at most hospitals requires the medical professional account for and dispose of the remaining unused medication by pouring the medication down a sink drain or into a toilet. Typically, another medical professional must observe the disposal and sign a statement that the medication was disposed of.

The current system of medication disposal is flawed because most prescription drugs are clear liquids. If a medical professional, being defined as a doctor, nurse, or any other person involved with handling and/or wasting narcotics, is a prescription drug abuser, that medical professional may wish to steal the medication. The drug abusing medical professional may secretly pour the medication into another container and fill the original container with water. Under the current system, the drug abusing medical professional can then find another medical professional to observe the disposal. The observing medical professional will see the disposing medical professional disposing of a clear liquid and assume the liquid is the medication when said liquid is in fact water. The disposing medical professional can the fraudulently fill out the appropriate paperwork showing the medication was properly disposed of. In this manner, medical professional with drug abuse problems or the desire to make illegal prescription drug sales can have easy access to powerful narcotics, and the current medication disposal system cannot detect that the medical professional has stolen the narcotics. Additionally, even if the drug is not clear due to mixture with some other compound or some other reason, a trained medical professional may still think of many ways of disguising a commonly available liquid as a narcotic and using this method of deceiving the current medication disposal system.

Therefore, what is needed is an apparatus for indicating the presence of a controlled substance and method of use. The apparatus should indicate when a given medication is present. The method of use should expose medical professional attempting to steal prescription medication, discourage other medical professional from stealing medication, and provide additional proof that non-drug abusing medical professional are not stealing medication. Furthermore, other desirable features and characteristics of the present invention will become apparent when this background of the invention is read in conjunction with the subsequent detailed description of the invention, appended claims, and the accompanying drawings.

### SUMMARY OF THE INVENTION

The present invention advantageously fills the aforementioned deficiencies by providing an apparatus for indicating the presence of a controlled substance that can indicate the presence of different types of powerful narcotics depending on the embodiment. The associated method of use, when adopted as a policy, will prove conclusively that the drugs being disposed have not been switched. The method of use will expose prescription drug thieves, protect innocent medical professional, and discourage would be prescription drug thieves from taking illegal actions.

In one particular embodiment of the present invention, a medical professional administers a dose of an opiate to a patient. The medical professional draws a small amount of the unused opiate into a syringe. The medical professional then punctures the cover of the apparatus and injects the opiate sample into the receptacle in the presence of another medical professional. The opiate then reacts chemically with the indicator in the receptacle and causes a color change. The remaining unused opiate can then be destroyed, and both medical professionals can attest to the fact that the destroyed material was the opiate and not some other compound.

In another embodiment, a medical professional administers a dose of a ketamine to a patient. If medical professional is a drug abuser, and wishes to steal the ketamine. The medical professional pours the ketamine into an extra bottle, hides the ketamine, and fills the original bottle with water. The medical professional then draws a small amount of the water into a syringe. The medical professional then punctures the cover of the apparatus and injects the opiate sample into the receptacle in the presence of another medical professional. The indicator in the receptacle does not react with the water and no color change occurs. The observing medical professional is then alerted that the drug abusing medical professional has stolen the ketamine and may take appropriate action.

In still another embodiment, a medical professional administers a dose of a benzodiazepine, to a patient. In the presence of another medical professional, the administering medical professional then opens the cover of the apparatus and pours a small amount of the remaining benzodiazepine into a receptacle and closes the apparatus. The benzodiazepine then reacts chemically with the indicator in the receptacle and causes a color change. The remaining unused benzodiazepine can then be destroyed, and both medical professionals can attest to the fact that the destroyed material was the benzodiazepine and not some other compound.

The present invention now will be described more fully hereinafter with reference to the accompanying drawings, which are intended to be read in conjunction with both this summary, the detailed description, and any preferred and/or particular embodiments specifically discussed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

The drawings contained herein exemplify one of the embodiments of the claimed invention. It should be noted that the invention is not limited to the embodiment shown. The embodiment shown is purely an example, and the invention is capable of many variations of said embodiment. In the drawings,

FIG. 1 illustrates a top view of the apparatus for indicating the presence of a controlled substance.

FIG. 2 illustrates a side cross-sectional view of the apparatus with a syringe injecting test material into the receptacle of the apparatus.

FIG. 3 illustrates a perspective view of the apparatus for indicating the presence of a controlled substance.

The first digit of each reference numeral in the above figures indicates the figure in which an element or feature is most prominently shown. The second digit indicates related elements or features, and a final letter (when used) indicates a sub-portion of an element or feature.

### DETAILED DESCRIPTION OF THE INVENTION

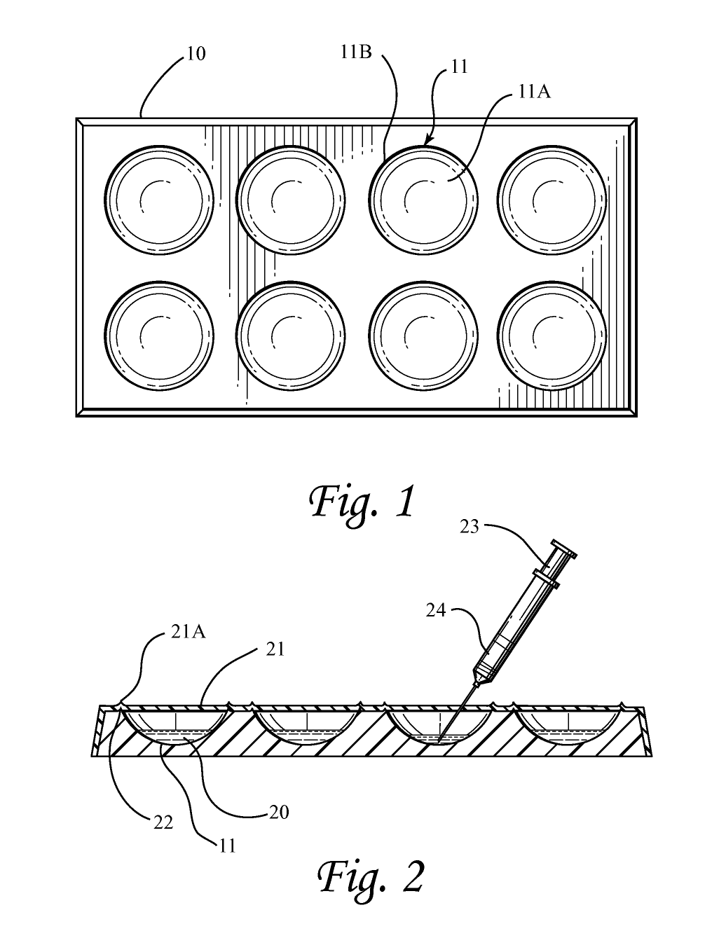

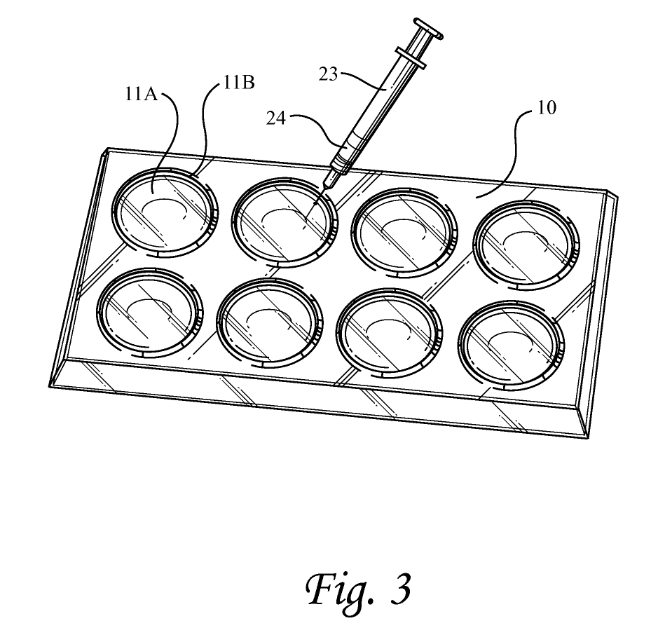

FIG. 1 illustrates a top view of the apparatus for indicating the presence of a controlled substance. The apparatus comprises a container element 10. The container element 10 should be comprised of, or coated with a material that does not react chemically with the indicator element 20 selected for a given embodiment as discussed below.

The container element 10 further comprises a plurality of receptacle elements 11. The purpose of the receptacle elements is to contain the indicator elements 20 as discussed below. The present embodiment comprises eight receptacle elements 11 positioned in two rows. However, the apparatus may comprise as many or as few receptacle elements 11 as desired for a given application. Additionally, the receptacle elements 11 may be arranged in any configuration desired as long as liquid placed in one receptacle element 11 will remain separated from liquid placed in other receptacle elements 11.

The receptacle elements 11 each further comprise an opening 11A. An opening 11A may be used to place materials into a given receptacle element 11. Each opening further comprises an edge 11B. An edge 11B is the boundary of an opening 11A.

FIG. 2 illustrates a side cross-sectional view of the apparatus for indicating the presence of a controlled substance. Each receptacle element 11 contains an indicator element 20. The indicator element 20 is pre-selected to cause a visible chemical reaction when placed in contact with a specific controlled substance. A medical professional can place a substance inside a receptacle element 11 of the container element 10 and in contact with an indicator element 20. If the substance placed in contact with the indicator element 20 comprises the expected controlled substance, the indicator element 20 will react chemically. The medical professional can then observe the reaction and ensure that the test substance does in fact comprise the controlled substance.

In one of the preferred embodiments, all of the indicator elements 20 comprise a Marquis reagent further comprising a mixture of formaldehyde and sulfuric acid. Marquis Reagents are known in the art to cause a color changing reaction when mixed with narcotics including but not limited to opiates, phenethylamines, cocaine, and Ecstasy. In this embodiment, the apparatus for indicating the presence of a controlled substance is designed to detect the presence of one or more of these compounds.

In another embodiment, all of the indicator elements 20 comprise a Zimmerman-Reinhardt reagent further comprising a mixture of nitrobenzene and methanol. Zimmerman-Reinhardt reagents are known in the art to cause a color changing reaction when mixed with narcotics including but not limited to ketamines and benzodiazepines. In this embodiment, the apparatus for indicating the presence of a controlled substance is designed to detect the presence of one or more of these compounds. In another embodiment, some of the indicator elements 20 comprise a Zimmerman-Reinhardt reagent while other indicator elements comprise a Marquis reagent. In still another embodiment, the indicator elements 20 are composed of any mixture that changes color in the presence of a specific controlled substance, including but not limited to mixtures containing cobalt thiocyanate and sodium hydroxide.

A cover element 21 is affixed to the container element 10 to prevent the indicator elements 20 from being released from the apparatus. The cover element 21 should be comprised of, or coated with a material that does not react chemically with the indicator element 20 selected for a given embodiment. The cover element further comprises a plurality of separation elements 21A. Each separation element 21A is affixed to the edge 11B of the opening 11A of a receptacle 11. Each separation element 21A is affixed an edge 11B to prevent the indicator element 20 contained in the receptacle 11 from mixing with the indicator elements 20 contained in other receptacles 11.

In the preferred embodiment, each edge 11B further comprises a separation element 22. In this case, the separation element 22 of the receptacle element 11 is affixed to the separation element 21A of the cover element 21. This configuration further decreases the likelihood of indicator element 20 mixing between receptacles 11.

In the preferred embodiment, the cover element 21 is permanently affixed to the container element 10 in the manner described above at the time the apparatus is manufactured. In this embodiment, the indicator element 20 is positioned inside the receptacle element 11 before the cover element 21 and the container element 10 are affixed.

In this embodiment, the cover element 21 comprises a material that can be punctured by a hand held syringe 23. In this manner, a medical professional may inject a material 24 to be tested for the presence of a controlled substance into the receptacle element 11. The material 24 will then be in contact and mix with the indicator element 20.

In another embodiment, the cover element 21 is removably affixed to the container element 10 in the manner described above at the time the apparatus is manufactured. In this embodiment, the indicator element 20 may be positioned inside the receptacle element 11 at any time after manufacture but before use. In this embodiment, the user may remove the cover element 21 from the container element 10 prior to placing the material 24 in contact with the indicator element 20, causing the material 24 to mix with said indicator element 20. The mixing may be accomplished by injecting, pouring, or any other standard clinical procedure. The embodiment with the removably affixed cover element 21 may also be used to test materials 24 that cannot be easily injected with a syringe 23.

A medical professional may use the apparatus when said professional needs to dispose of a material 24 containing a controlled substance. The medical professional may take a portion of the material 24 and inject or otherwise place the material 24 into the receptacle element 11 and in contact with the indicator element 20 as discussed above and allow the two compounds to mix. The medical professional may then observe the mixture for signs of a reaction prior to disposing of the remainder of the material 24. If a visible reaction occurs, the medical professional may then record the results, and dispose of the apparatus when all of the indicator elements 11 have been utilized. In this manner, the medical professional has obtained verifiable proof that the material 24 disposed of contained the controlled substance. However, if the medical professional has switched the controlled substance with another similar substance in an attempt to steal the controlled substance, the medical professional will be caught immediately and may be reprimanded appropriately.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

I claim:

1. An apparatus for indicating the presence of a controlled substance comprising,
a plurality of indicator elements for indicating the presence of a controlled substance;
a container element further comprising a plurality of receptacle elements, for containing the indicator elements, each receptacle element further comprising an opening, each opening further comprising an edge; and
a cover element for preventing the release of the indicator elements, further comprising a plurality of separation elements;
wherein each indicator element is positioned inside a receptacle element of the container element, and
each separation element of the cover element is affixed to the edge of the opening of a receptacle element of the container element.

2. The apparatus of claim 1, wherein each edge of each opening of each receptacle element further comprises a separation element affixed to a separation element of the cover element.

3. The apparatus of claim 2, wherein each receptacle element is comprised of a material that does not chemically react with the indicator element positioned inside said receptacle element.

4. The apparatus of claim 2, wherein each receptacle element is coated with a material that does not chemically react with the indicator element positioned inside said receptacle element.

5. The apparatus of claim 2, wherein the cover element is comprised of a material that does not chemically react with the indicator element positioned inside an affixed receptacle element.

6. The apparatus of claim 2, wherein the cover element is coated with a material that does not chemically react with the indicator element positioned inside an affixed receptacle element.

7. The apparatus of claim 2, wherein each separation element of the cover element is permanently affixed to each separation element of edge of each opening of each receptacle element of the container element.

8. The apparatus of claim 7, wherein the cover element is comprised of a material which can be pierced by a hand operated syringe.

9. The apparatus of claim 2, wherein each separation element of the cover element is removably affixed to each separation element of edge of each opening of each receptacle element of the container element.

10. The apparatus of claim 2, wherein a plurality of indicator elements comprise any chemical that visibly reacts with an opiate.

11. The apparatus of claim 2, wherein a plurality of indicator elements comprise any chemical that visibly reacts with a benzodiazepine.

12. The apparatus of claim 2, wherein a plurality of indicator elements comprise any chemical that visibly reacts with a ketamine.

13. The apparatus of claim 2, wherein a plurality of indicator elements comprise a Marquis reagent.

14. The apparatus of claim 2, wherein a plurality of indicator elements comprise a Zimmerman-Reinhardt reagent.

15. A method for determining whether a material contains a controlled substance comprising;
operating an apparatus for indicating the presence of a controlled substance;
said apparatus comprising;
a plurality of indicator elements for indicating the presence of a controlled substance;
a container element further comprising a plurality of receptacle elements, for containing the indicator elements, each receptacle element further comprising an opening, each opening further comprising an edge; and
a cover element for preventing the release of the indicator elements, further comprising a plurality of separation elements;
wherein each indicator element is positioned inside a receptacle element of the container element, and
each separation element of the cover element is affixed to the edge of the opening of a receptacle element of the container element;
said operation comprising;
placing a substance inside a receptacle element of the container element and in contact with an indicator element; and
observing the indicator element and substance mixture for signs of a chemical reaction.

16. The method of claim 15, wherein placing a material inside a receptacle element of the container element and in contact with an indicator element is accomplished by injecting the substance into the receptacle element with a syringe.

17. The method of claim 15, further comprising removing the cover element before placing a substance inside a receptacle element of the container element and in contact with an indicator element.

18. The method of claim 15, further comprising recording whether a chemical reaction has occurred after placing the substance inside a receptacle element of the container element and in contact with an indicator element.

19. The method of claim 18, further comprising disposing of the remaining controlled substance after recording whether a chemical reaction has occurred.

20. The method of claim 19, further comprising disposing of the apparatus after all of the indicator elements in all of the receptacle elements have been placed in contact with a material.
